(19) United States
(12) Patent Application Publication
(10) Pub. No.: US 2020/0046906 A1
(43) Pub. Date: Feb. 13, 2020

(54) Injection Device Having a Capability of Controlling Injection Dose

(71) Applicant: CC Biotechnology Corporation, Tainan City (TW)

(72) Inventor: Chin-Min Yeh, Tainan City (TW)

(21) Appl. No.: 16/487,935

(22) PCT Filed: Feb. 24, 2017
(86) PCT No.: PCT/CN2017/074771

§ 371 Date: Aug. 22, 2019

(51) Int. Cl. A61M 5/315; A61M 5/20

(52) U.S. Cl. CPC A61M 5/31536; A61M 5/2033; A61M 5/31551

(57) ABSTRACT

An injection device has a dose controlling assembly, an operation assembly, an injection pushing assembly, and a clutch assembly. The dose controlling assembly has an outer tube, a dose controlling tube, a driving tube, a connection tube, and a dose controlling collar. The driving tube has a limiting portion. The dose controlling collar is mounted in the dose controlling tube and has a spiral limiting portion selectively engaged with the limiting portion of the driving tube. The operation assembly is mounted on a rear end of the dose controlling assembly. The injection pushing assembly is mounted on a front end of the dose controlling assembly. The clutch assembly is mounted in a front segment of the outer tube and is connected with the injection pushing assembly.

### BACKGROUND OF THE INVENTION

### 1. Field of the Invention

The present invention relates to an injection device, and more particularly to an injection pen that can control the injection dose and can be automatically locked after predetermined shots of injection.

### 2. Description of Related Art

For safety, an injection device can only be used for pre-determined times of shots. A conventional injection device includes two types, wherein one type is quantitative type and the other is time-metering type. In a conventional injection device of the quantitative type, after the injection device is connected with a medication vial, a pushing rod mounted on a rear end of the injection device is pushed. Consequently, a piston in the medication vial is pushed to move for a predetermined distance with the transmission of a driving assembly of the injection device. Accordingly, a fixed amount of medication is injected into a patient.

However, the driving assembly of the conventional injection device still works and is not locked after the medication in the vial is used up. Therefore, the injection device may be inadvertently applied to inject different medications to different persons. Therefore, the safety of using the conventional injection device is insufficient.

To overcome the shortcomings, the present invention tends to provide an injection device to mitigate or obviate the aforementioned problems.

### SUMMARY OF THE INVENTION

The main objective of the invention is to provide an injection device that can control the injection dose and can be automatically locked after predetermined times of injection.

To achieve the objective, the present invention provides an injection device having a dose controlling assembly, an operation assembly, an injection pushing assembly, and a clutch assembly.

The dose controlling assembly comprises
an outer tube having
   an axial hole defined axially through the outer tube; and
   a spiral rib formed on an inner surface of the axial hole of the outer tube;
a dose controlling tube mounted rotatably in the axial hole in the outer tube and comprising
   an axial hole defined axially through the dose controlling tube;
   a front segment having a spiral groove engaged with the spiral rib of the outer tube;
   a rear segment formed at a rear end of the dose controlling tube; and
   multiple engaging grooves defined longitudinally in an inner surface at the rear end of the axial hole of the dose controlling tube;
a driving tube mounted in the axial hole in the dose controlling tube and having
   a front segment;
   a rear segment formed at a rear end of the driving tube;
   a first inner space defined in the front segment of the driving tube and having an opening at a front end of the driving tube;
   a second inner space defined in the rear segment of the driving tube and having an opening at the rear end of the driving tube;
   at least one notch defined radially in the rear segment of the driving tube and communicating with the second inner space, and each one of the at least one notch having an engaging tab formed in the notch and selectively engaged with the engaging grooves in the dose controlling tube; and
   a limiting portion formed on the front end of the driving tube;
a connection tube mounted in the dose controlling tube and having
   a rear end connected with the front end of the driving tube;
   an axial hole defined axially through the connection tube;
   a connection segment provided with a thread and having a front end and a rear end; and
   an annular flange formed around the front end of the connection segment;
a dose controlling collar mounted in the dose controlling tube, located in front of the driving tube, and having
   a threaded hole threaded with the thread on the connection segment of the connection tube; and
   a rear end provided with a spiral limiting portion selectively engaged with the limiting portion of the driving tube;

an operation assembly mounted on a rear end of the dose controlling assembly and comprising
an operation element mounted in the second inner space of the driving tube to push the engaging tab on the driving tube to engage with the engaging grooves in the dose controlling tube and having
   an insertion sheet extending outside the rear end of the driving tube, selectively extending into the rear end of the dose controlling tube, and having a front end and a rear end; and
   an insertion head formed on the rear end of the insertion sheet;
a compression spring mounted around the insertion sheet of the operation element and having two ends respectively abutting the rear end of the driving tube and the insertion head; and
a pushing element mounted around the insertion head of the operation element;

an injection pushing assembly mounted on a front end of the dose controlling assembly and comprising
an axial tube having
   an axial hole defined axially through the axial tube;
   a tube body mounted in the connection tube, being axially moveable relative to the connection tube, and having a front end; and
   a front segment formed on the front end of the tube body and located in front of the connection tube and the dose controlling tube;
a pushing rod mounted in the axial hole in the axial tube, being axially moveable relative to and driven to rotate with the axial tube, and having a front end and a pushing thread formed around the pushing rod; and
a pushing block mounted on the front end of the pushing rod; and

a clutch assembly mounted in a front segment of the outer tube, connected with the injection pushing assembly, and comprising
a front casing mounted securely in a front end of the outer tube and having
   a rear board having a threaded hole defined through the rear board and threaded with the pushing thread on the pushing rod; and
   a first ratchet portion formed on a rear side of the rear board and around the threaded hole of the rear board and having multiple unidirectional ratchet teeth arranged annularly;
a rear casing mounted securely in the front end of the outer tube and connected with a rear end of the front casing;
a clutch element mounted moveably in the rear casing, being driven to rotate with the axial tube, and having
   a central hole formed axially through the clutch element and disposed moveably around the front segment of the axial tube; and
   a second ratchet portion formed on a front side of the clutch element and having multiple unidirectional ratchet teeth arranged annularly and selectively engaged with the multiple unidirectional ratchet teeth of the first ratchet portion on the front casing; and
a pushing spring mounted in the rear casing, mounted around the front segment of the axial tube, and having two ends respectively abutting the clutch element and the axial tube.

Wherein, the dose controlling tube further has multiple engaging teeth formed around the inner surface at the rear end of the axial hole of the dose controlling tube; and

the driving tube further has multiple resilient arms respectively engaged with the engaging teeth in the dose controlling tube.

Wherein, the outer tube further has a limiting groove formed in the inner surface of the axial hole at a rear end of the outer tube; and

the dose controlling tube further has a limiting protrusion formed on an outer surface at a position between the front segment and the rear segment of the dose controlling tube and selectively engaged with the limiting groove in the outer tube.

Wherein, the dose controlling tube further has a resilient hook formed on a front end of the front segment of the dose controlling tube to engage with the connection tube and to hold the connection tube in place.

Wherein, the driving tube further has at least one engaging hole defined in the front segment of the driving tube;

the connection tube further has an assembling segment connected with the rear end of the connection segment and having
multiple slits formed longitudinally in the assembling segment and extending to a rear end of the assembling segment; and
at least one engaging block formed on an outer surface of the assembling segment and engaged respectively with the at least one engaging hole in the driving tube.

Wherein, the engaging tab in each one of the at least one notch of the driving tube has
a holding block formed on the engaging tab; and
a limiting block formed on the engaging tab and spaced from the holding block; and

the operation element further has a pushing head formed on the front end of the insertion sheet, held between the holding block and the limiting block of the engaging tab of the driving tube, and having a rear side selectively abutting on and limited by the limiting block of the engaging tab.

Wherein, the thread on the connection segment of the connection tube has a pitch smaller than a thread pitch of the spiral groove in the dose controlling tube; and

a pitch of the pushing thread on the pushing rod is smaller than the thread pitch of the spiral groove in the dose controlling tube and is larger than the pitch of the thread on the connection segment of the connection tube.

Wherein, the pushing rod has at least one rod guiding groove defined longitudinally in an outer surface of the pushing rod and extending through the pushing thread on the pushing rod;

the axial tube further has at least one guiding rib formed longitudinally on an inner surface of the axial hole of the axial tube and engaged respectively with the at least one rod guiding groove in the pushing rod;

the front segment of the axial tube has at least one front guiding groove formed longitudinally in an outer surface of the front end of the axial tube;

the axial tube further has at least one rear guiding groove formed longitudinally in an outer surface of the axial tube;

the connection tube further has at least one connection guiding rib formed longitudinally on an inner surface of the axial hole of the connection tube and engaged respectively with the at least one rear guiding groove in the axial tube;

the dose controlling tube further has at least one controlling rib formed longitudinally on an inner surface of the axial hole of the dose controlling tube; and

the connection tube further has at least one recess defined longitudinally in an outer surface of the annular flange of the connection tube and engaged respectively with the at least one controlling rib on the dose controlling tube.

Wherein, the axial tube further has an annular protrusion formed around the axial tube at a position between the tube body and the front segment of the axial tube and located in front of the connection tube and the dose controlling tube;

the rear casing comprises
a tubular body having a rear end and a chamber defined in the tubular body; and
a rear board formed on the rear end of the tubular body and having a through hole defined through the rear board of the rear casing and communicating with the chamber;

the tube body extends through the through hole in the rear casing;

the front segment and the annular protrusion of the axial tube are held in the chamber in the rear casing;

the rear board of the rear casing is located between the annular protrusion of the axial tube and the dose controlling tube;

the clutch element and the pushing spring are held in the chamber of the rear casing; and

one of the ends of the pushing spring abuts the annular protrusion on the axial tube.

With the arrangement of the injection device comprising the dose controlling assembly, an operation assembly, an injection pushing assembly, and a clutch assembly, the injection device in accordance with the present invention can control the injection dose in each injection process and is automatically locked by the engagement between the spiral portion of the dose controlling collar and the limiting portion of the driving tube after the injection device has been used the predetermined times. Therefore, the used injection device cannot be used repeatedly, and the safety of using the injection device is improved.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of an injection device in accordance with the present invention;

FIG. 2 is another exploded perspective view of the injection device in FIG. 1,

FIG. 3 is a cross sectional side view of the injection device in FIG. 1;

FIG. 4 is an enlarged exploded perspective view of the injection pushing assembly of the injection device in FIG. 1;

FIG. 5 is another enlarged exploded perspective view of the injection pushing assembly of the injection device in FIG. 1;

FIG. 6 is an enlarged perspective view of the clutch assembly of the injection device in FIG. 1 in an engaged status;

FIG. 7 is an enlarged perspective view of the clutch assembly of the injection device in FIG. 1 in a released status;

FIG. 8 is an operational cross sectional side view of the injection device in FIG. 1 with a protection cap and a needle cap;

FIG. 9 is an operational cross sectional side view of the injection device in FIG. 8 with the protection cap and the needle cap being removed;

FIG. 10 is an enlarged operational cross sectional side view of the injection device in FIG. 9;

FIG. 11 is an enlarged operational cross sectional side view of the injection device in FIG. 9 with the operation assembly being pulled backward;

FIG. 12 is an enlarged operational cross sectional side view of the injection device in FIG. 9 with the operation assembly being pushed frontward; and

FIG. 13 is an enlarged operational cross sectional side view of the injection device in FIG. 9 in a locked status.

### DETAILED DESCRIPTION OF PREFERRED EMBODIMENT

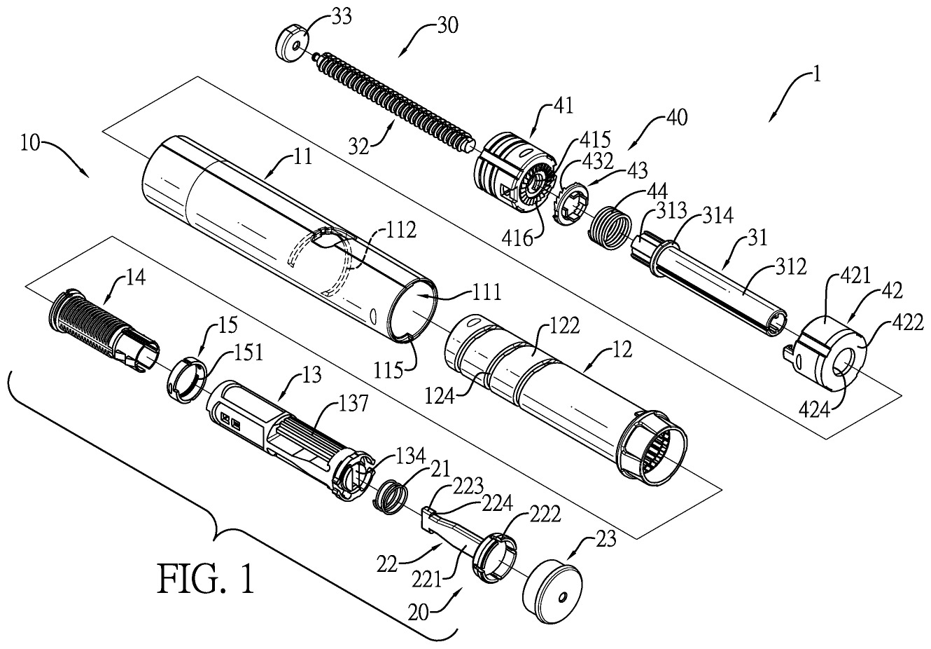

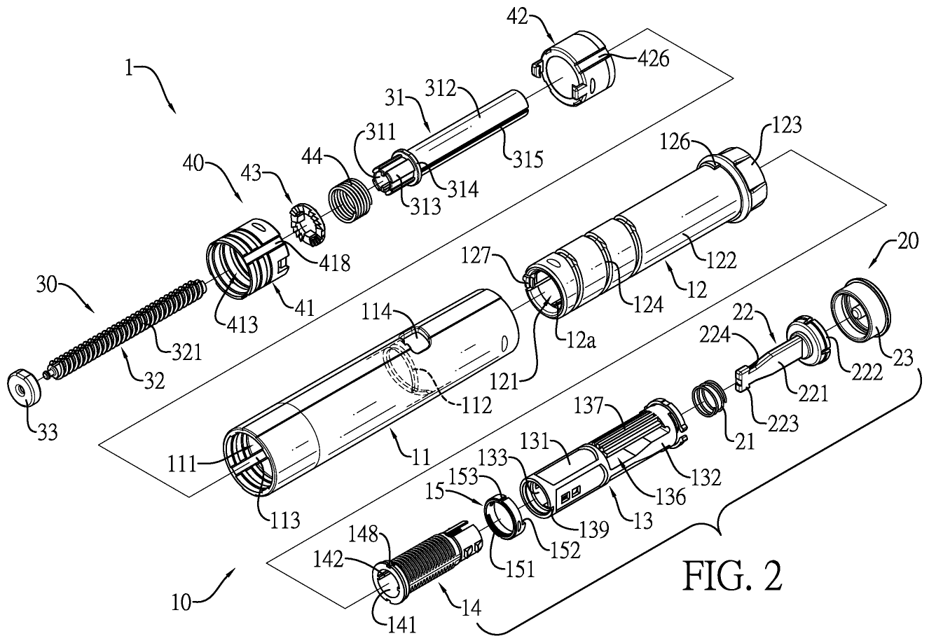

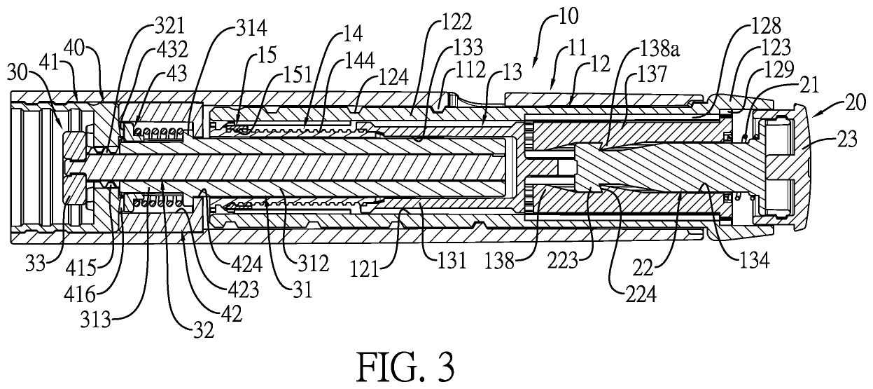

With reference to FIGS. 1 to 3, an injection device in accordance with the present invention comprises a dose controlling assembly 10, an operation assembly 20, an injection pushing assembly 30, and a clutch assembly 40.

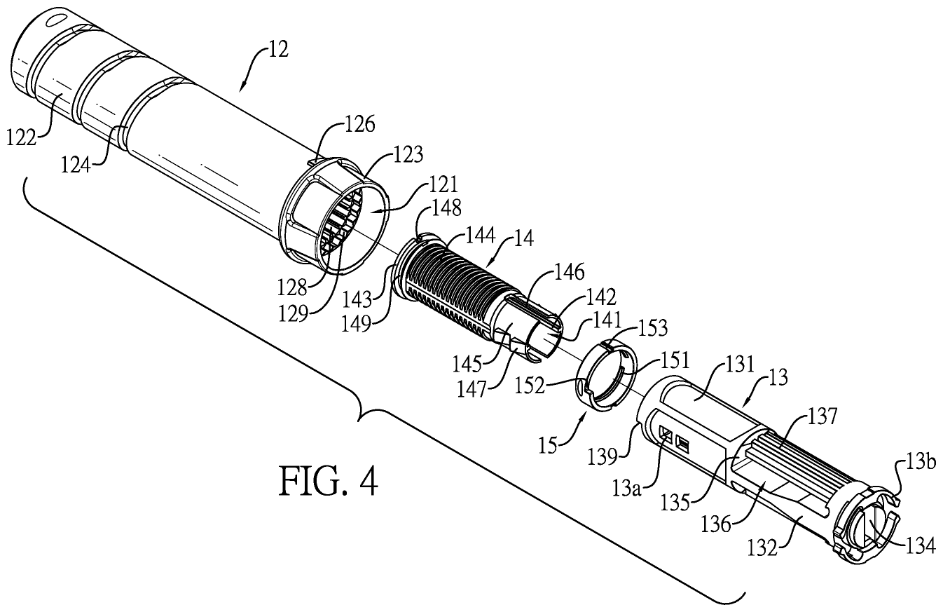

With reference to FIGS. 1 to 4, the dose controlling assembly 10 comprises an outer tube 11, a dose controlling tube 12, a driving tube 13, a connection tube 14, and a dose controlling collar 15.

The outer tube 11 has an axial hole 111, a spiral rib 112, at least one holding rib 113, a through hole 114, and a limiting groove 115. The axial hole 111 is defined axially through the outer tube 11. The spiral rib 112 is formed on an inner surface of the axial hole 111 of the outer tube 11. The at least one holding rib 113 is formed longitudinally on the inner surface of the axial hole 111 near a front end of the outer tube 11. Alternatively, the outer tube 11 further has multiple annular channels defined in the inner surface of the axial hole 111 near the front end of the outer tube 11. The through hole 114 is defined radially in the outer tube 11 and communicates with the axial hole 111. The limiting groove 115 is formed in the inner surface of the axial hole 111 at a rear end of the outer tube 11.

The dose controlling tube 12 is mounted rotatably in the axial hole 111 in the outer tube 11 and comprises an axial hole 121, a front segment 122, a rear segment 123, a resilient hook 127, multiple controlling ribs 12a, multiple engaging grooves 128, and multiple engaging teeth 129. The axial hole 121 is defined axially through the dose controlling tube 12. The front segment 122 is formed at the front end of the dose controlling tube 12 and has a spiral groove 124 formed on an outer surface of the front segment 122 and engaged with the spiral rib 112 of the outer tube 11. The rear segment 123 is formed at a rear end of the dose controlling tube 12. Preferably, a limiting protrusion 126 is formed on an outer surface of the dose controlling tube 12 at a position between the front segment 122 and the rear segment 123 and selectively engaged with the limiting groove 115 in the outer tube 11. The resilient hook 127 is formed on a front end of the front segment 122 of the dose controlling tube 12. Multiple controlling ribs 12a are formed longitudinally on an inner surface of the axial hole 121 of the dose controlling tube 12. The engaging grooves 128 are defined longitudinally in the inner surface at the rear end of the axial hole 121 of the dose controlling tube 12. The engaging teeth 129 are formed around the inner surface at the rear end of the axial hole 121 of the dose controlling tube 12.

The driving tube 13 is mounted in the axial hole 121 in the dose controlling tube 12 and comprises a front segment 131, a rear segment 132, a first inner space 133, a second inner space 134, at least one notch 136, multiple resilient arms 13b, and a limiting portion 139. The front segment 131 is formed at a front end of the driving tube 13, and the rear segment 132 is formed at a rear end of the driving tube 13. The first inner space 133 is defined in the front segment 131 of the driving tube 13 and has an opening at the front end of the driving tube 13. The second inner space 134 is defined in the rear segment 132 of the driving tube 13 and has an opening at the rear end of the driving tube 13. The at least one notch 136 is defined radially in the rear segment 132 of the driving tube 13 and communicates with the second inner space 134. Each one of the at least one notch 136 has an engaging tab 137 formed in the notch 136 and can be radially pushed to selectively engage with the engaging grooves 128 in the dose controlling tube 12. Each engaging tab 137 has a holding block 138 and a limiting block 138a. The holding block 138 is formed on the engaging tab 137. The limiting block 138a is formed on the engaging tab 137 and is spaced from the holding block 138. The resilient arms 13b are formed on a rear end of the rear segment 132 and are respectively engaged with the engaging teeth 129 in the dose controlling tube 12. The limiting portion 139 is formed on the front end of the driving tube 13. Preferably, the driving tube 13 may further have at least one engaging hole 13a defined in the front segment 131 of the driving tube 13. The second inner space 134 is rectangular in cross section.

The connection tube 14 is mounted in the dose controlling tube 12 and has a front end, a rear end, an axial hole 141, at least one connection guiding rib 142, an annular flange 143, an annular groove 149, at least one recess 148, a connection segment 144, and an assembling segment 145. The rear end of the connection tube 14 is connected with the front end of the driving tube 13 and is limited by the resilient hook 127 of the dose controlling tube 12. The axial hole 141 is defined axially through the connection tube 14. The at least one connection guiding rib 142 is formed longitudinally on an inner surface of the axial hole 141 of the connection tube 14. The annular flange 143 is formed around the front end of the connection tube 14. The annular groove 149 is defined around an outer surface of the annular flange 143. The at least one recess 148 is defined longitudinally in the outer surface of the annular flange 143 and is engaged respectively with the at least one controlling rib 12a on the dose controlling tube 12. The connection segment 144 is provided with a thread and has a front end and a rear end. The assembling segment 145 is connected with the rear end of the connection segment 144 and has multiple slits 146 and at least one engaging block 147. The slits 146 are formed longitudinally in the assembling segment 145 and extend to a rear end of the assembling segment 145. The at least one engaging block 147 is formed on an outer surface of the assembling segment 145 and is engaged respectively with the at least one engaging hole 13a in the driving tube 13. With the engagement of the at least one engaging block 147 and the at least one engaging hole 13a, the connection tube 14 is connected with the front segment 131 of the driving tube 13.

The dose controlling collar 15 is mounted in the dose controlling tube 12, is located in front of the driving tube 13, is screwed with the connection segment 144 of the connection tube 14, and has a threaded hole 151, a rear end, and at least one recess 153. The threaded hole 151 is defined in the dose controlling collar 15 and is threaded with the thread on the connection segment 144 of the connection tube 14. The rear end of the dose controlling collar 15 is provided with a spiral limiting portion 152 selectively engaged with the limiting portion 139 of the driving tube 13. The at least one recess 153 is defined longitudinally in an outer surface of the dose controlling collar 15 and is engaged respectively with at least one of the controlling ribs 12a on the dose controlling tube 12 to allow the dose controlling collar 15 to rotate with the dose controlling tube 12. At an original position, the dose controlling collar 15 is located at the front end of the connection segment 144 of the connection tube 14. The travel of the dose controlling collar 15 is stopped when the spiral portion 152 is engaged with the limiting portion 139 of the driving tube 13.

With reference to FIGS. 1 to 3, the operation assembly 20 is mounted on a rear end of the dose controlling assembly 10 and comprises an operation element 22, a compression spring 21, and a pushing element 23. The operation element 22 is mounted in the second inner space 134 of the driving tube 13 to push the engaging tab 137 on the driving tube 13 to engage with the engaging grooves 128 in the dose controlling tube 12 and has an insertion sheet 221, a pushing head 223, and an insertion head 222. The insertion sheet 221 extends outside the rear end of the driving tube 13, selectively extends into the rear end of the dose controlling tube 12, and has a front end and a rear end. The pushing head 223 is formed on the front end of the insertion sheet 221 and is held between the holding block 138 and the limiting block 138a of the engaging tab 137 of the driving tube 13. The pushing head 223 has a rear side selectively abutting on and limited by the limiting block 138a of the engaging tab 137 to prevent the operation element 22 from being detached from the driving tube 13 when the operation element 22 is pulled backward. The insertion head 222 is formed on the rear end of the insertion sheet 221.

The compression spring 21 is mounted around the insertion sheet 221 of the operation element 22 and has two ends respectively abutting the rear end of the driving tube 13 and the insertion head 222. The pushing element 23 is mounted around the insertion head 222 of the operation element 22, is rotatable and axially moveable relative to the operation element 22, and is capable of extending into the rear end of the dose controlling tube 12.

In the operation assembly 20, the operation element 22 can be pushed to move forward, and the pushing head 223 pushes against the holding block 138 on the engaging tab 137 of the driving tube 13, and the engaging tab 137 is pushed to radially move outward and engage the engaging grooves 128 in the dose controlling tube 12. Consequently, the driving tube 13 is connected with the dose controlling tube 12. When the operation element 22 is pulled backward, the engaging tab 137 radially moves inward and disengages from the engaging grooves 128 in the dose controlling tube 12.

The injection pushing assembly 30 is mounted on a front end of the dose controlling assembly 10 and comprises an axial tube 31, a pushing rod 32, and a pushing block 33. The axial tube 31 has an axial hole 311, a tube body 312, an annular protrusion 314, and a front segment 313. The axial hole 311 is defined axially through the axial tube 31. The tube body 312 is mounted in the connection tube 14, is axially moveable relative to the connection tube 14, and has a front end. The front segment 313 is formed on the front end of the tube body 312 and is located in front of the connection tube 14 and the dose controlling tube 12. The annular protrusion 314 is formed around the axial tube 31 at a position between the tube body 312 and the front segment 313 of the axial tube 31 and is located in front of the connection tube 14 and the dose controlling tube 12.

The pushing rod 32 is mounted in the axial hole 311 in the axial tube 31, is axially moveable relative to and driven to rotate with the axial tube 31, and has a front end and a pushing thread 321 formed around the pushing rod 32. The pushing thread 321 has a pitch smaller than a thread pitch of the spiral groove 124 on the dose controlling tube 12 but larger than a pitch of the thread of the connection segment 144 of the connection tube 14. The pushing block 33 is mounted on the front end of the pushing rod 32 and is located in front of the axial tube 31.

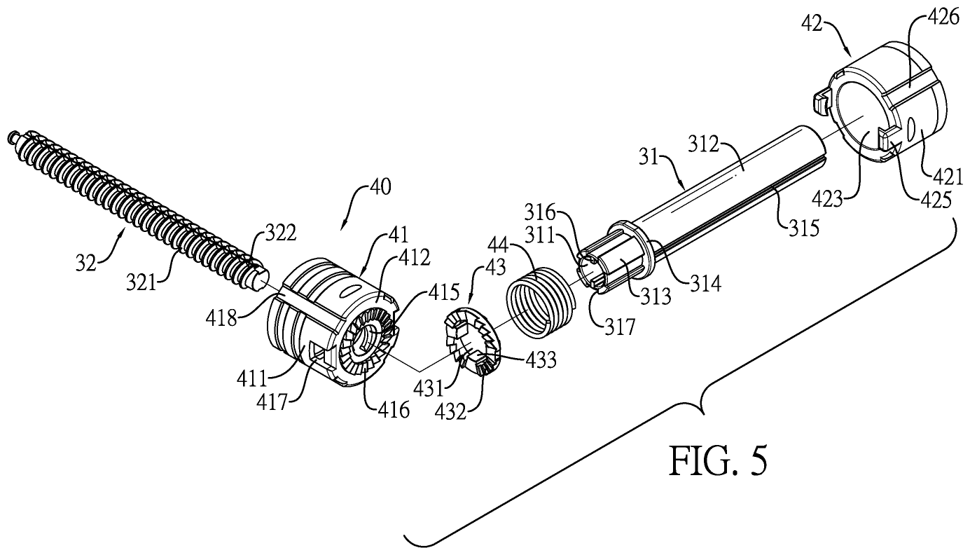

With reference to FIGS. 1 to 3 and 5, the pushing rod 32 further has at least one rod guiding groove 322 defined longitudinally in an outer surface of the pushing rod 32 and extending through the pushing thread 321 on the pushing rod 32. The axial tube 31 further has at least one guiding rib 316 formed longitudinally on an inner surface of the axial hole 311 of the axial tube 31 and engaged respectively with the at least one rod guiding groove 322 in the pushing rod 32. Consequently, the pushing rod 32 is axially moveable relative to the axial tube 31. The front segment 313 of the axial tube 31 further has at least one front guiding groove 317 formed longitudinally in an outer surface of the front end of the axial tube 31. The axial tube 31 further has at least one rear guiding groove 315 formed longitudinally in an outer surface of the axial tube 31 and engaged respectively with the connection guiding rib 142 in the axial hole 141 of the connection tube 14.

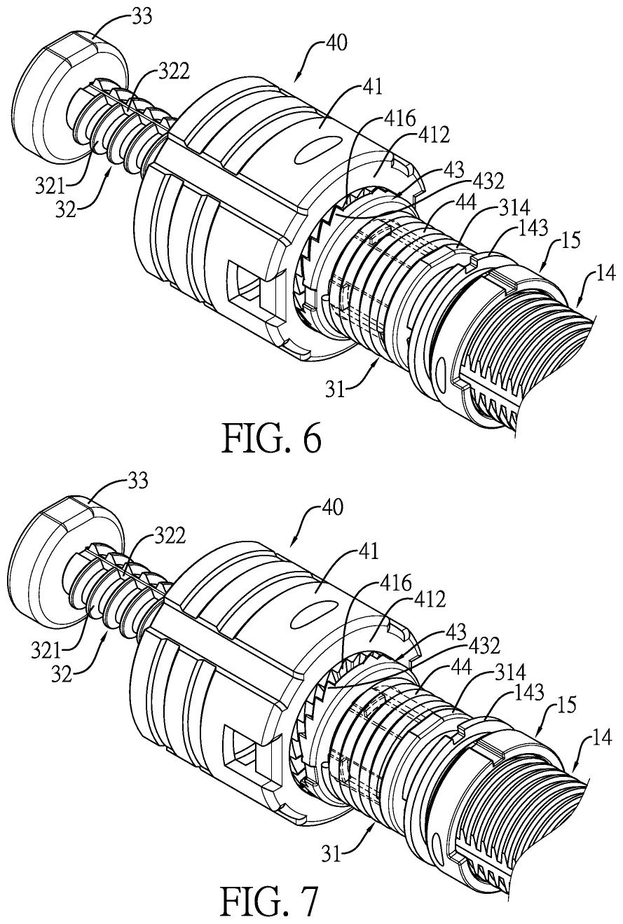

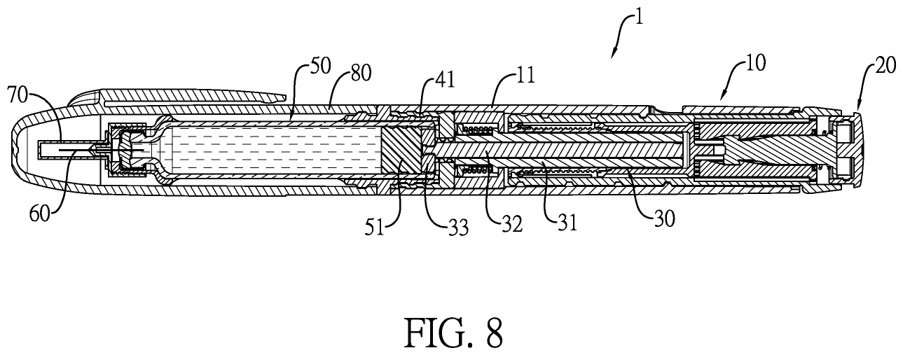

With reference to FIGS. 1 to 3 and 5 to 7, the clutch assembly 40 is mounted in the front end of the outer tube 11, is connected with the injection pushing assembly 30, and comprises a front casing 41, a rear casing 42, a clutch element 43, and a pushing spring 44. The front casing 41 is mounted securely in the front end of the outer tube 11 and has at least one combination groove 418, a casing body 411, a rear board 412, and a first ratchet portion 416. The at least one combination groove 418 is defined respectively in an outer surface of the front casing 41. Alternatively, the front casing 41 may further have multiple annular flanges. When the front casing 41 is mounted in the axial hole 111 of the outer tube 11, the at least one combination groove 418 is engaged with the at least one holding rib 113 and the annular flanges are engaged with the annular channels, such that the front casing 41 is mounted securely in the outer tube 11. The casing body 411 has a chamber 413 provided with an opening defined in a front end of the casing body 411. The rear board 412 is formed on a rear end of the casing body 411 and has a threaded hole 415. The threaded hole 415 is defined through the rear board 412, communicates with the chamber 413, and is threaded with the pushing thread 321 on the pushing rod 32. The first ratchet portion 416 is formed on a rear side of the rear board 412 and around the threaded hole 415 of the rear board 412 and has multiple unidirectional ratchet teeth arranged annularly.

The rear casing 42 is mounted securely in the front end of the outer tube 11 and is connected with a rear end of the front casing 41. Preferably, multiple hooks 425 and holes are defined between the front casing 41 and the rear casing 42 to connect the front casing 41 with the rear casing 42. The rear casing 42 may have multiple combination grooves 426 defined longitudinally in an outer surface of the rear casing 42 and engaged respectively with the at least one holding rib 113 in the outer tube 10. Consequently, the rear casing 42 is mounted securely in the axial hole 111 in the outer tube 11. The rear casing 42 has a casing body 421 and a rear board 422. The casing body 421 has a chamber 423 provided with an opening defined in a front end of the casing body 422. The rear board 422 has a through hole 424 defined through the rear board 422 and communicating with the chamber 423. The tube body 312 of the axial tube 31 is mounted through the through hole 424 in the rear board 422. The front segment 313 and the annular protrusion 314 of the axial rube 31 are held in the chamber 423 of the rear casing 42. The rear board 422 is located between the annular protrusion 314 and the dose controlling tube 12.

The clutch element 43 is mounted moveably in the chamber 423 of the rear casing 42, is mounted axially moveably around the front segment 313 of the axial tube 31, is driven to rotate with the axial tube 31, and has a central hole 431 and a second ratchet portion 432. The central hole 431 is formed axially through the clutch element 43 and is mounted moveably around the front segment 313 of the axial tube 31. Preferably, the central hole 431 has at least one guiding protrusion 433 formed longitudinally on an inner surface of the central hole 431 and engaged respectively in the at least one front guiding groove 317 in the axial tube 31, such that the clutch element 43 is axially moveable relative to and is rotated with the axial tube 31. The second ratchet portion 432 is formed on a front side of the clutch element 43 and has multiple unidirectional ratchet teeth arranged annularly and selectively engaged with or disengaged from the multiple unidirectional ratchet teeth of the first ratchet portion 416 on the front casing 41.

The pushing spring 44 is mounted in the chamber 423 of the rear casing 42, is mounted around the front segment 313 of the axial tube 31, and has two ends respectively abutting the clutch element 43 and the annular protrusion 314 of the axial tube 31 to provide a resilient force to the clutch element 43. With the resilient force provided by the pushing spring 44, the second ratchet portion 432 of the rear casing 42 is engaged with the first ratchet portion 416 of the front casing 41.

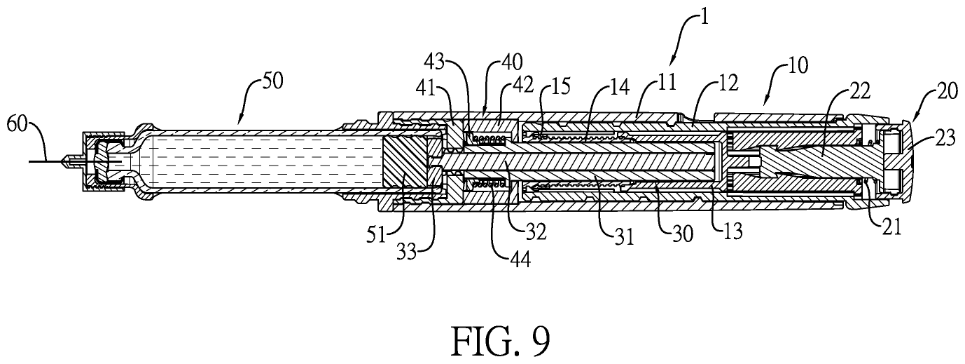

With reference to FIGS. 8 and 9, the medication vial 50 is connected with the casing body 411 of the front casing 41 of the clutch assembly 40, and the pushing block 33 of the pushing rod 32 extends into the medication vial 50 and abuts a piston 51. A needle 60 is mounted on a front end of the medication vial 50, and a needle cap 70 is mounted around the needle 60. In addition, a protection cap 80 is mounted around the needle cap 70 and the medication vial 50. The protection cap 80 has a rear end mounted around the casing body 411 of the front casing 41.

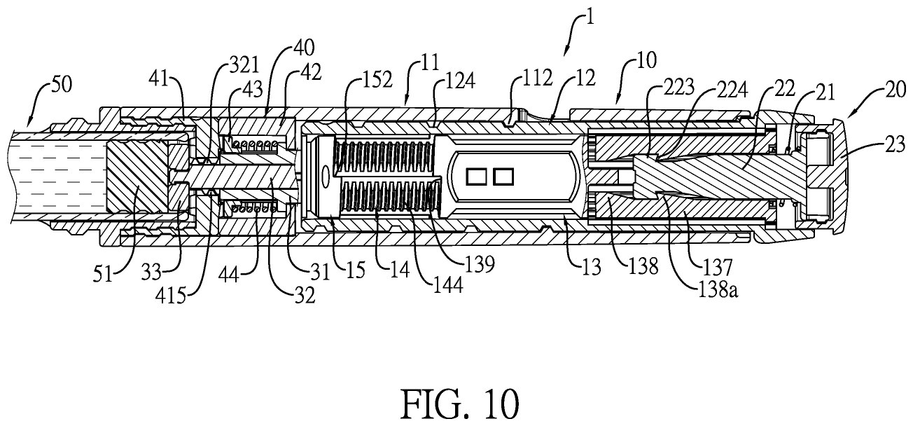

With reference to FIGS. 9 to 12, in use, the protection cap 80 and the needle cap 70 are removed first, and the needle 60 is inserted into the body of a patient. The pushing element 23 is pulled backward to extend out of the rear end of the outer tube 11 for a length. At this time, the front casing 41 and the rear casing 42 are mounted securely in the outer tube 11, and the first ratchet portion 416 on the front casing 41 is engaged with the second ratchet portion 432 on the clutch element 43 and the axial tube 31 is prevented from being rotated. Consequently, the connection tube 14 is only allowed to move backward due to the limiting effect provided by the axial tube 31 to the driving tube 13 and the connection tube 14. With the dose controlling tube 12 moved backward with the driving tube 13 and the connection tube 14, the dose controlling tube 12 is rotated backward relative to the outer tube 11 in a predetermined number of circles due to the spiral connection between the outer tube 11 and the dose controlling tube 12. With the rotation of the dose controlling tube 12, the dose controlling collar 15 is also rotated and moved relative to the connection tube 14 to move backward. The resilient arms 13b on the driving tube 13 will generate sound when engaged with the engaging teeth 129 on the dose controlling tube 12.

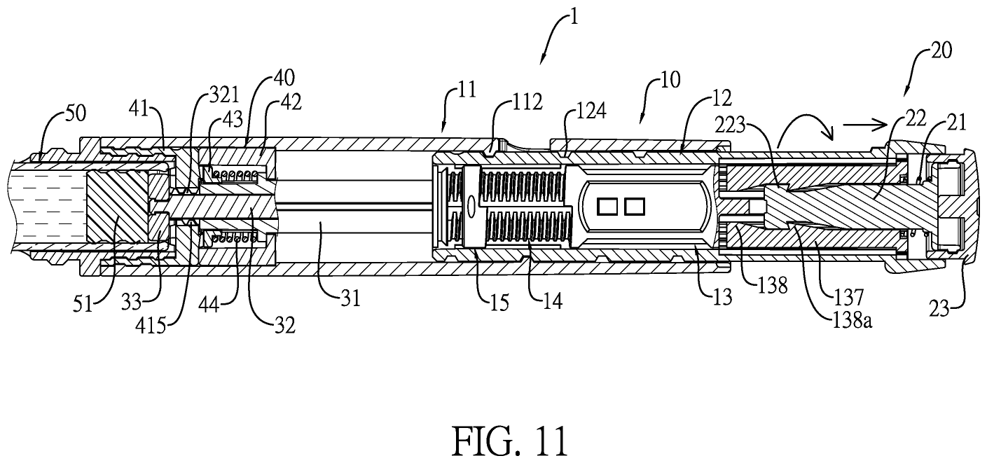

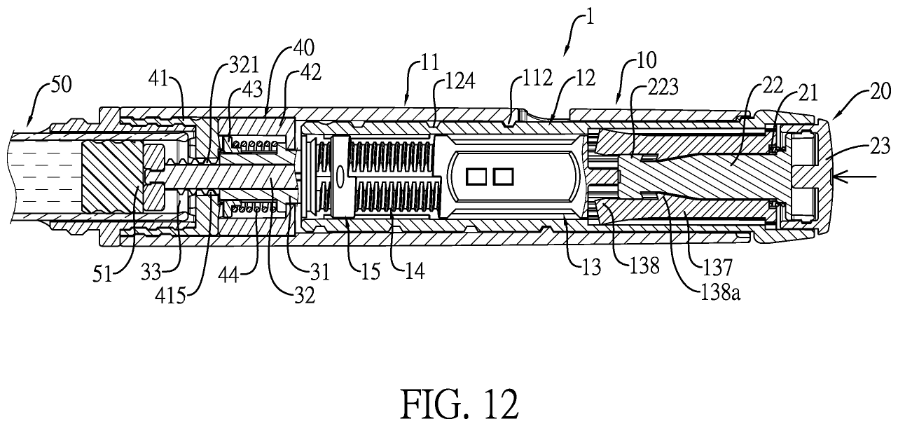

With reference to FIGS. 11 and 12, when the pushing element 23 is pushed forward, the insertion sheet 221 of the operation element 22 is moved forward to push the resilient tab 137 on the driving tube 13 to engage with the engaging grooves 128 in the dose controlling tube 12. Consequently, the driving tube 13 and the dose controlling tube 12 are connected with each other. With the spiral connection between the outer tube 11 and the dose controlling tube 12, the driving tube 13, the dose controlling tube 12, the connection tube 14 and the dose controlling collar 15 can be rotated and moved relative to the axial tube 31. In addition, the driving tube 13 can drive the axial tube 31 to rotate at a fixed position. Because the first ratchet portion 416 and the second ratchet portion 432 have the unidirectional ratchet teeth, the clutch element 43 is rotated with the axial tube 31 while the axial tube 31 is rotated. With the inclined surfaces of the unidirectional teeth pushing each other, the pushing spring 44 is compressed and the second ratchet portion 432 is disengaged from the first ratchet portion 416. The clutch element 43 can be rotated with the axial tube 31, and the pushing rod 32 can be driven to rotate by the axial tube 31. With the threaded connection between the pushing rod 32 and the front casing 41, the rotating pushing rod 32 can be moved to push the piston 51 in the medication vial 50 to be moved by the pushing block 33. Accordingly, a certain amount of medication can be injected into the patient via the needle 60.

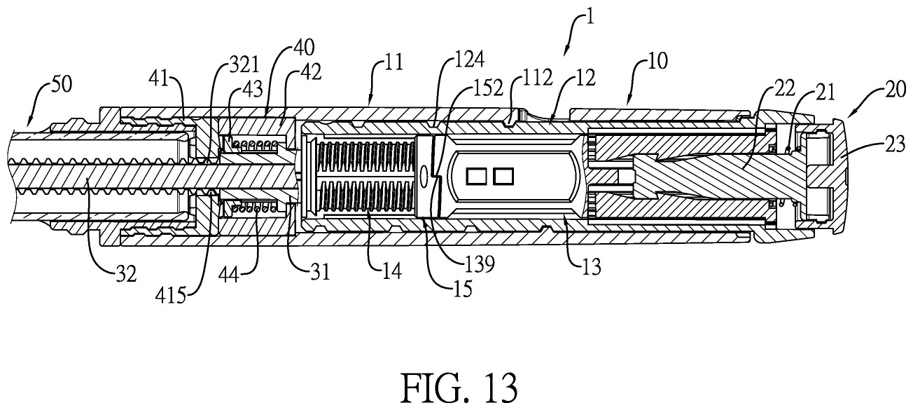

The injection device in accordance with the present invention can be used to inject medication for a certain times of shots. To improve the safety of using the injection device, a used needle 60 should be removed and discarded after each shot. In each injection process, when the operation assembly 20 is pulled backward, the dose controlling collar 15 will move along the connection tube 14 toward the front end of the driving tube 13 for a certain distance. The certain distance of the movement of the dose controlling collar 15 is determined by the times of injection of the injection device, the amount of medication of each injection and the total amount of the medication in the vial 50. With reference to FIG. 13, after the dose controlling collar 15 is driven to rotate backward several times, the spiral portion 152 of the dose controlling collar 15 will be engaged with the limiting portion 139 of the driving tube 13. After the pushing element 23 of the operation element 20 is pushed for injecting medication at the final injection process, the dose controlling collar 15 is connected securely with the driving tube 13, and the driving tube 13 is kept from being rotated due to the engagement between the front casing 41 and the clutch element 43. The dose controlling tube 12 is limited by the dose controlling collar 15 and is kept from being rotated backward. At this time, the medication in the vial 50 is used up. The injection device in accordance with the present invention is automatically locked and cannot be used repeatedly.

The injection device in accordance with the present invention can control the injection dose in each injection process and is automatically locked by the engagement between the spiral portion 152 of the dose controlling collar 15 and the limiting portion 139 of the driving tube 13 after the injection device being used the predetermined times. Therefore, the used injection device cannot be used repeatedly, and the safety of using the injection device is improved.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. An injection device comprising:
a dose controlling assembly comprising:
an outer tube having
an axial hole defined axially through the outer tube; and
a spiral rib formed on an inner surface of the axial hole of the outer tube;
a dose controlling tube mounted rotatably in the axial hole in the outer tube and comprising
an axial hole defined axially through the dose controlling tube;
a front segment having a spiral groove engaged with the spiral rib of the outer tube;
a rear segment formed at a rear end of the dose controlling tube; and
multiple engaging grooves defined longitudinally in an inner surface at a rear end of the axial hole of the dose controlling tube;
a driving tube mounted in the axial hole in the dose controlling tube and having
a front segment;
a rear segment formed at a rear end of the driving tube;
a first inner space defined in the front segment of the driving tube and having an opening at a front end of the driving tube;
a second inner space defined in the rear segment of the driving tube and having an opening at the rear end of the driving tube;
at least one notch defined radially in the rear segment of the driving tube and communicating with the second inner space, and each one of the at least one notch having an engaging tab formed in the notch and selectively engaged with the engaging grooves in the dose controlling tube; and
a limiting portion formed on the front end of the driving tube;
a connection tube mounted in the dose controlling tube and having
a rear end connected with the front end of the driving tube;
an axial hole defined axially through the connection tube;
a connection segment provided with a thread and having a front end and a rear end; and
an annular flange formed around the front end of the connection segment;
a dose controlling collar mounted in the dose controlling tube, located in front of the driving tube, and having
a threaded hole threaded with the thread on the connection segment of the connection tube; and
a rear end provided with a spiral limiting portion selectively engaged with the limiting portion of the driving tube;
an operation assembly mounted on a rear end of the dose controlling assembly and comprising
an operation element mounted in the second inner space of the driving tube to push the engaging tab on the driving tube to engage with the engaging grooves in the dose controlling tube and having
an insertion sheet extending outside the rear end of the driving tube, selectively extending into the rear end of the dose controlling tube, and having a front end and a rear end; and
an insertion head formed on the rear end of the insertion sheet;
a compression spring mounted around the insertion sheet of the operation element and having two ends respectively abutting the rear end of the driving tube and the insertion head; and
a pushing element mounted around the insertion head of the operation element;
an injection pushing assembly mounted on a front end of the dose controlling assembly and comprising
an axial tube having
an axial hole defined axially through the axial tube;
a tube body mounted in the connection tube, being axially moveable relative to the connection tube, and having a front end; and
a front segment formed on the front end of the tube body and located in front of the connection tube and the dose controlling tube;
a pushing rod mounted in the axial hole in the axial tube, being axially moveable relative to and driven to rotate with the axial tube, and having a front end and a pushing thread formed around the pushing rod; and
a pushing block mounted on the front end of the pushing rod; and
a clutch assembly mounted in a front segment of the outer tube, connected with the injection pushing assembly, and comprising
a front casing mounted securely in a front end of the outer tube and having
a rear board having a threaded hole defined through the rear board and threaded with the pushing thread on the pushing rod; and
a first ratchet portion formed on a rear side of the rear board and around the threaded hole of the rear board and having multiple unidirectional ratchet teeth arranged annularly;
a rear casing mounted securely in the front end of the outer tube and connected with a rear end of the front casing;
a clutch element mounted moveably in the rear casing, being driven to rotate with the axial tube, and having
a central hole formed axially through the clutch element and disposed moveably around the front segment of the axial tube; and
a second ratchet portion formed on a front side of the clutch element and having multiple unidirectional ratchet teeth arranged annularly and selectively engaged with the multiple unidirectional ratchet teeth of the first ratchet portion on the front casing; and
a pushing spring mounted in the rear casing, mounted around the front segment of the axial tube, and having two ends respectively abutting the clutch element and the axial tube.

2. The injection device as claimed in claim 1, wherein
the dose controlling tube further has multiple engaging teeth formed around the inner surface at the rear end of the axial hole of the dose controlling tube; and
the driving tube further has multiple resilient arms respectively engaged with the engaging teeth in the dose controlling tube.

3. The injection device as claimed in claim 2, wherein
the outer tube further has a limiting groove formed in the inner surface of the axial hole at a rear end of the outer tube; and
the dose controlling tube further has a limiting protrusion formed on an outer surface at a position between the front segment and the rear segment of the dose controlling tube and selectively engaged with the limiting groove in the outer tube.

4. The injection device as claimed in claim 2, wherein the dose controlling tube further has a resilient hook formed on a front end of the front segment of the dose controlling tube to engage with the connection tube and to hold the connection tube in place.

5. The injection device as claimed in claim 4, wherein
the driving tube further has at least one engaging hole defined in the front segment of the driving tube;
the connection tube further has an assembling segment connected with the rear end of the connection segment and having
multiple slits formed longitudinally in the assembling segment and extending to a rear end of the assembling segment; and
at least one engaging block formed on an outer surface of the assembling segment and engaged respectively with the at least one engaging hole in the driving tube.

6. The injection device as claimed in claim 5, wherein
the engaging tab in each one of the at least one notch of the driving tube has
a holding block formed on the engaging tab; and
a limiting block formed on the engaging tab and spaced from the holding block; and
the operation element further has a pushing head formed on the front end of the insertion sheet, held between the holding block and the limiting block of the engaging tab of the driving tube, and having a rear side selectively abutting on and limited by the limiting block of the engaging tab.

7. The injection device as claimed in claim 5, wherein
the thread on the connection segment of the connection tube has a pitch smaller than a thread pitch of the spiral groove in the dose controlling tube; and
a pitch of the pushing thread on the pushing rod is smaller than the thread pitch of the spiral groove in the dose controlling tube and is larger than the pitch of the thread on the connection segment of the connection tube.

8. The injection device as claimed in claim 6, wherein
the thread on the connection segment of the connection tube has a pitch smaller than a pitch of the spiral groove in the dose controlling tube; and
the pushing thread on the pushing rod is smaller than the pitch of the spiral groove in the dose controlling tube and is larger than the pitch of the thread on the connection segment of the connection tube.

9. The injection device as claimed in claim 8, wherein
the pushing rod has at least one rod guiding groove defined longitudinally in an outer surface of the pushing rod and extending through the pushing thread on the pushing rod;
the axial tube further has at least one guiding rib formed longitudinally on an inner surface of the axial hole of the axial tube and engaged respectively with the at least one rod guiding groove in the pushing rod;
the front segment of the axial tube has at least one front guiding groove formed longitudinally in an outer surface of the front end of the tube body of the axial tube;
the axial tube further has at least one rear guiding groove formed longitudinally in an outer surface of the axial tube;
the connection tube further has at least one connection guiding rib formed longitudinally on an inner surface of the axial hole of the connection tube and engaged respectively with the at least one rear guiding groove in the axial tube;
the dose controlling tube further has at least one controlling rib formed longitudinally on an inner surface of the axial hole of the dose controlling tube; and
the connection tube further has at least one recess defined longitudinally in an outer surface of the annular flange of the connection tube and engaged respectively with the at least one controlling rib on the dose controlling tube.

10. The injection device as claimed in claim 9, wherein
the axial tube further has an annular protrusion formed around the axial tube at a position between the tube body and the front segment of the axial tube and located in front of the connection tube and the dose controlling tube;
the rear casing comprises
a tubular body having a rear end and a chamber defined in the tubular body; and
a rear board formed on the rear end of the tubular body and having a through hole defined through the rear board of the rear casing and communicating with the chamber;
the tube body extends through the through hole in the rear casing;
the front segment and the annular protrusion of the axial tube are held in the chamber in the rear casing;
the rear board of the rear casing is located between the annular protrusion of the axial tube and the dose controlling tube;
the clutch element and the pushing spring are held in the chamber of the rear casing; and
one of the ends of the pushing spring abuts the annular protrusion on the axial tube.

11. The injection device as claimed in claim 1, wherein
the engaging tab in each one of the at least one notch of the driving tube has
a holding block formed on the engaging tab; and
a limiting block formed on the engaging tab and spaced from the holding block; and
the operation element further has a pushing head formed on the front end of the insertion sheet, held between the holding block and the limiting block of the engaging tab of the driving tube, and having a rear side selectively abutting on and limited by the limiting block of the engaging tab.

12. The injection device as claimed in claim 1, wherein
the thread on the connection segment of the connection tube has a pitch smaller than a thread pitch of the spiral groove in the dose controlling tube; and
a pitch of the pushing thread on the pushing rod is smaller than the thread pitch of the spiral groove in the dose controlling tube and is larger than the pitch of the thread on the connection segment of the connection tube.

13. The injection device as claimed in claim 11, wherein
the thread on the connection segment of the connection tube has a pitch smaller than a pitch of the spiral groove in the dose controlling tube; and
the pushing thread on the pushing rod is smaller than the pitch of the spiral groove in the dose controlling tube and is larger than the pitch of the thread on the connection segment of the connection tube.

14. The injection device as claimed in claim 13, wherein
the pushing rod has at least one rod guiding groove defined longitudinally in an outer surface of the pushing rod and extending through the pushing thread on the pushing rod;
the axial tube further has at least one guiding rib formed longitudinally on an inner surface of the axial hole of the axial tube and engaged respectively with the at least one rod guiding groove in the pushing rod;
the front segment of the axial tube has at least one front guiding groove formed longitudinally in an outer surface of the front end of the tube body of the axial tube;
the axial tube further has at least one rear guiding groove formed longitudinally in an outer surface of the axial tube;
the connection tube further has at least one connection guiding rib formed longitudinally on an inner surface of the axial hole of the connection tube and engaged respectively with the at least one rear guiding groove in the axial tube;
the dose controlling tube further has at least one controlling rib formed longitudinally on an inner surface of the axial hole of the dose controlling tube; and
the connection tube further has at least one recess defined longitudinally in an outer surface of the annular flange of the connection tube and engaged respectively with the at least one controlling rib on the dose controlling tube.

15. The injection device as claimed in claim 14, wherein
the axial tube further has an annular protrusion formed around the axial tube at a position between the tube body and the front segment of the axial tube and located in front of the connection tube and the dose controlling tube;
the rear casing comprises
a tubular body having a rear end and a chamber defined in the tubular body; and
a rear board formed on the rear end of the tubular body and having a through hole defined through the rear board of the rear casing and communicating with the chamber;
the tube body extends through the through hole in the rear casing;
the front segment and the annular protrusion of the axial tube are held in the chamber in the rear casing;
the rear board of the rear casing is located between the annular protrusion of the axial tube and the dose controlling tube;
the clutch element and the pushing spring are held in the chamber of the rear casing; and
one of the ends of the pushing spring abuts the annular protrusion on the axial tube.
